(19) United States
(12) Patent Application Publication
(10) Pub. No.: US 2026/0283467 A1
(43) Pub. Date: Sep. 24, 2026

(54) MECHANISMS AND METHODS FOR CONTROLLED VOLUME INJECTION INTO A SOFT MEDIA WITH CONCURRENT PRESSURE MEASUREMENT

(71) Applicant: MASSACHUSETTS INSTITUTE OF TECHNOLOGY, Cambridge, MA (US)

(72) Inventors: Brendan M. UNIKEWICZ, Cambridge, MA (US); Emily R. SOLOGUREN, Cambridge, MA (US); Joseph P. MORALES, Yonkers, NY (US); André Milo PINCOT, Southern Pines, NC (US); Sarah A. SAMS, Parker, CO (US); Nevan Clancy HANUMARA, Cambridge, MA (US); Alexander H. SLOCUM, Bow, NH (US); Tal COHEN, Cambridge, MA (US)

(21) Appl. No.: 19/475,576

(22) PCT Filed: May 15, 2024
(86) PCT No.: PCT/US2024/029550

§ 371 Date: Oct. 15, 2025

Related U.S. Application Data
(60) Provisional application No. 63/502,411, filed on May 15, 2023.

(51) Int. Cl. A61B 5/00; A61M 5/28; A61M 5/36

(52) U.S. Cl. CPC A61B 5/0048; A61M 5/282; A61M 5/36

(57) ABSTRACT

Systems and devices capable of precise delivery of fluid, on the order of microliters and below, into a tissue or soft material, while concurrently measuring fluidic pressure in order to generate a pressure-volume curve from which material properties can be determined. Specifically, examples include systems and devices designed to conduct digital palpation of a tissue by measuring internal mechanical resistance using fluid delivered by a needle placed in the tissue. Examples include the use of the measurement of mechanical resistance, as well as other features, such as fracture strength, to differentiate tissue types, or tissue health, such as cancerous versus non-cancerous.

### CROSS-REFERENCE TO RELATED APPLICATION

The present disclosure claims priority to and the benefit of U.S. Provisional Application No. 63/502,411, entitled “MECHANISMS FOR CONTROLLED VOLUME INJECTION INTO A SOFT MEDIA WITH CONCURRENT PRESSURE MEASUREMENT,” filed on May 15, 2023, the content of which is incorporated by reference herein in its entirety.

### FIELD

The present disclosure relates to devices, systems, and methods for controlled injection of a volume of fluid into a soft media against of pressure with concurrent pressure measurement to determine physical properties of the soft media.

### BACKGROUND

Tissue types can often be differentiated through differences in stiffness. In the case, for example, of skin cancer diagnosis, manual palpation of the skin can identify regions of different stiffness, although the measurement is qualitative and resolution, accuracy and reliability are limited. In the case of internal tissues, there is no way to measure quantitatively stiffness in a minimally invasive manner. In the industrial material science scenario, there exists a wide way to measure stiffness, including tensile and indention tests and needle induced cavitation rheology. While applicable to excised ex-vivo tissue, these tests can be destructive and are not designed or physically suited to be conducted upon living tissue in-vivo.

There are wide ranges of cancers that present as discrete, identifiable nodules, such as breast, lung and thyroid; however, these can also be resultant from other pathologies, such as cysts, localized infections and benign pathologies. It would be desirable to have a minimally invasive means to measure stiffness and other material properties of these nodules, as well as other tissues, as a means of differentiating different pathologies. Fine needle and core biopsies can be conducted to acquire samples of cells or bulk tissue, respectively, for pathological analysis, however, the stiffness of the limited size samples cannot be determined.

As an example, approximately 1.2% of people will develop thyroid cancer during their lifetime, and prevalence of the disease is rising. Manual palpation, which is possible due to the thyroid's accessible location, and ultrasound of the thyroid are common practices to identify, but not qualify nodules. Subsequent diagnostic methods for thyroid cancer include fine-needle aspiration (FNA) biopsy and cytological molecular testing. The rate of incorrect FNA results for thyroid nodules was reported to be 2%-10% and 3.6%-10.2% respectively, and the resulting overtreatment due to indeterminate results leads to excessive and unnecessary thyroidectomy procedures. Improving preoperative differentiation of thyroid and other potentially cancerous nodules would substantially decrease the possibility of conducting unnecessary invasive surgeries.

Improving in-vivo, real-time differentiation and diagnostics by measuring tissue stiffness, regardless of depth, would significantly reduce the cost burden of diagnosing cancers and other pathologies, reduce medical providers' reliance on off-site laboratories for analysis of potentially cancerous tissue and improve patient care and experience.

Volume-controlled cavity expansion (VCCE) has been validated as a method for determining material properties of soft media. VCCE involves expanding a cavity in a soft material by injecting a volume of an incompressible fluid into the material. The resulting expansion of the material by the fluid allows numerous mechanical properties of the media to be observed. Additionally, constitutive material models may be applied to the result of the volumetric expansion of the cavity and the resisting pressure of the tissue. At the micro- and nano-liter scale, injecting a predetermined volume of fluid into resisting soft tissue of millimeter and centimeter scale, is challenging with issues affecting measurement accuracy, including bubbles, material compliance, and stiction. Prior devices for accurately dispensing a micro to nano volume of fluid include syringes with actuated plungers and micro-pumps. Generally, air bubbles are removed by priming a syringe, inverting it, and expelling fluid, but while practical for some systems, micro-bubbles can remain clinging to surfaces. Additional compliance and control challenges can come from mechanical plunger seals and the syringe body itself. Additionally, standard syringes, having fixed chamber widths, pose limitations in volume control—leaving all system precision required to be addressed in the actuation mechanisms. Although micro-pumps offer the necessary aforementioned precision, they are expensive, large, exhibit stiction and other usability issues complicating diagnostic utility.

Therefore, there is a need for improved systems and devices to precisely dispense a micro volume of fluid into soft media while counteracting resisting pressure from soft media to provide minimally invasive methods for measuring the mechanical properties of tissue.

### SUMMARY

The present application is directed to injection systems and devices capable of precise delivery of fluid, on the order of microliters and below, into a tissue or soft material, while concurrently measuring fluidic pressure in order to generate a pressure-volume curve from which material properties can be determined. Specifically, examples of the present disclosure include systems and devices that can conduct “digital palpation” of a tissue by measuring internal mechanical resistance using fluid delivered by a needle placed in the tissue. Examples of the present disclosure include measurement of mechanical resistance, and other features, such as fracture strength, and viscoelasticity, that can be used to differentiate tissue types, or tissue health, such as cancerous versus non-cancerous. Examples of the present disclosure include systems and methods that have applicability not only as diagnostic tools, but also in situations where it is desirable to precisely and incrementally dispense fluid volumes against a backpressure, especially in a small form factor.

One example of the present disclosure is an injection device that includes a tubular conduit having a deformable wall, the deformable wall defining a channel extending between a proximal end and a distal end of the tubular conduit. The deformable wall comprises six sidewalls arranged as a convex hexagon and joined at angles by flexible joint regions between each of the six sidewalls. The six sidewalls include opposing and parallel first and second primary sidewalls and two opposing pairs of secondary sidewalls, the side sidewalls defining an perimeter of the channel.

The injection device can include an inlet at a proximal end of the tubular conduit, an outlet at a distal end of the tubular conduit, and a needle fluidly coupled with the outlet and configured to pierce into a soft material and deliver fluid from the tubular conduit to the soft material. The injection device can further include an actuator comprising a first surface contacting the first primary sidewall of the tubular conduit and a second surface contacting a second sidewall of the tubular conduit. The actuator can be configured to move a distance to deflect the tubular conduit by moving the first surface towards the second surface such that the angles at the flexible joint regions adjust to reduce the distance between the first primary sidewall and second primary sidewall. In some examples, the tubular conduit is configured to maintain a constant circumference of the perimeter during deflection of the first and second sidewalls and upon deflection of the tubular conduit by the actuator, the outlet is configured to expel a micro-volume of an incompressible fluid from the channel against resisting pressure from the soft material.

In some embodiments, the actuator is configured to move the first surface and the second surface simultaneously to reduce the distance between the first primary sidewall and the second primary sidewall. The first surface can contact the entire length of the first primary sidewall and the second surface can also contact the entire length of the second primary sidewall. In some embodiments, the conduit can further include a rigid wall region at the distal end before the outlet.

In some embodiments, the injection device can further include a controller configured to control the distance of the actuator based on a predetermined relationship between a distance of actuator movement and a micro-volume of fluid displaced from the tubular conduit.

The injection device can further include a pressure sensor configured to measure pressure within the tubular conduit during an amount of time and the pressure within the tubular conduit can correspond to a resisting pressure of the material at the outlet. The resisting pressure at the outlet is related to an expansion of a body of fluid at the outlet and at least one elastic property of the material.

In some embodiments, the injection device can further include a second conduit coupled to the inlet and configured to fill the tubular conduit while eliminating air from the fluid. In some embodiments, the fluid exhibits laminar flow through the tubular conduit. For example, the ratio of a length of the conduit and an inner radius of the conduit is greater than the fluid Reynolds number divided by 48.

Another example of the present disclosure is a volume controlled injection system that includes a tubular conduit having a deformable wall, the deformable wall defining a central channel extending between first and second ends of the tubular conduit. The channel defines a volume suitable for containing a fluid therein and the volume of the channel decreases upon exterior compression of the deformable wall. The system can further include an actuator configured to compress the deformable wall by symmetrically impinging opposing sides of the deformable wall along an axis lateral to a central longitudinal axis of the tubular conduit and a controller configured to control movement of the actuator. The actuator is configured to move a distance to deflect the deformable wall toward the central longitudinal axis and reduce the volume of the channel such that a corresponding volume of fluid is displaced from the central channel through an outlet at the second end of the tubular conduit and the controller is configured to control the distance based on a predetermined relationship between actuator movement and a volume of fluid displaced from the tubular conduit.

In some embodiments, the tubular conduit maintains a constant circumference of a perimeter of the channel during deflection of the opposing sides. A change in volume of the channel can relate to the volume of fluid displaced from the tubular conduit.

In some embodiments, the system further includes a pressure transducer configured to measure pressure in the central channel and the pressure in the central channel corresponds to a resisting pressure of the material at the outlet of the tubular conduit.

In some embodiments, the system further includes a housing having an inner cavity configured to house the tubular conduit, a handle for manual control, and an opening covered by a spring-loaded cover.

In some embodiments, the actuator includes a first plate and a second plate disposed on opposing sides of the tubular conduit and a step motor configured to move the first plate towards the second plate to compress the tubular conduit between the first and second plates. In some embodiments, the actuator can further include a second pressure sensor configured to measure pressure resulting from contact of the actuator against the deformable wall. The second pressure sensor can be a limit switch configured to limit the amount of actuator movement when a desired degree of sidewall deformation is reached.

In certain embodiments, the tubular conduit further includes a region of high stiffness and a region of low stiffness and the actuator is configured to compress the region of low stiffness.

In some embodiments, the system further includes a power source configured to power the controller and the actuator.

In some embodiments, the volume of fluid displaced from the central channel is approximately in the range of about 1 nanoliter to about 1000 nanoliters. In other embodiments, the volume of fluid displaced from the central channel is approximately in the range of about 1 microliter to about 1000 microliters.

Examples of the present disclosure include methods for measuring elastic properties of soft material that include providing a conduit having a deformable wall defining a channel pre-filled with an incompressible fluid and inserting an outlet needle of the conduit into a soft material. The method further includes deflecting an exterior portion of the deformable wall by displacement of an actuator to reduce the inner volume of the channel and introduce a body of incompressible fluid into the soft material from the conduit over an operation period of time. An internal pressure of the conduit can be recorded over the operation period of time.

The method can further include determining an expansion of the body of incompressible fluid over the operation period of time based on a volume change of the body of incompressible fluid over the operation period of time and determining an elastic property of the soft material based on a predetermined relationship between a displacement distance of the actuator, the expansion of the body of incompressible fluid, and the internal pressure of the conduit during the operation period of time.

In some embodiments, the operation period of time extends from a start of actuator movement to at least a time when the body of incompressible fluid disperses into the soft material. In some embodiments, the internal pressure of the conduit corresponds to the pressure of the soft material at the outlet. In some embodiments, the deformable wall of the conduit is deflected symmetrically by the actuator.

In some embodiments, the deformable wall comprises a region of high stiffness and a region of low stiffness and the actuator is configured to effect the compression upon the region of low stiffness.

In some embodiments, the actuator comprises a first plate and a second plate disposed on opposing sides of the conduit and a step motor configured to move at least one of the first and second plates to compress the conduit between the first and second plates.

In some embodiments, the fluid is incompressible and devoid of air. The disclosed method can further comprise pre-filling the conduit by coupling a one-way valve at an inlet of the conduit to a second conduit filled with the incompressible fluid and the one-way valve is configured to eliminate bubbles from the conduit.

In some embodiments, the operation period of time is approximately in the range of about 0.01 seconds to about 1000 seconds. The material can a non-biological material, which can be a soft tissue including thyroid tissue. In some embodiments, the soft tissue is within a human patient.

Another example of the present disclosure is a method for measuring material properties of a soft material that includes receiving by a processor, a first input comprising an increase in volume of a body of fluid introduced into the soft material over an operation period of time and a second input comprising a resisting pressure of the soft material over the operation period of time. The body of fluid can be spherical and the method can further include determining a circumferential stretch of the body of fluid during the operation period of time based on the first input and determining at least one material property of the soft material based on the circumferential stretch and the second input. In some examples, the operation period of time is from an initiation of the increase in volume of the body of fluid until a time when the body of fluid disperses throughout the soft material. In some examples, the at least one material property includes at least one of elastic modulus, toughness, viscoelastic response or fracture progression. In some examples, the method includes displaying a visual representation of the resisting pressure over the operation of time and the at least one material property on a visual display.

Another example of the present disclosure is a system for measuring material properties of a soft material that includes a tubular conduit having a deformable wall, the wall defining a central channel extending between first and second ends of the tubular conduit. The channel defines a volume suitable for containing a fluid therein. The system includes an actuator configured to move a distance along an axis lateral to a central longitudinal axis of the tubular conduit to deflect the deformable wall toward the central longitudinal axis and reduce the volume of the channel such that a corresponding volume of fluid is displaced from an outlet of the tubular conduit and a controller configured to control movement of the actuator. The controller is configured to control the distance based on a predetermined relationship between actuator movement and a volume of fluid displaced from the tubular conduit. In some examples, the tubular conduit includes a rigid material with an opening exposing a deformable material. In some examples, the tubular conduit includes a rigid material over molded on a flexible material or a flexible material over molded on a rigid material. In some examples, the tubular conduit includes a depressed region configured to receive the actuator.

Any of the features or variations described herein can be applied to any particular aspect or embodiment of the present disclosure in a number of different combinations. The absence of explicit recitation of any particular combination is due solely to avoiding unnecessary length or repetition.

### BRIEF DESCRIPTION OF THE DRAWINGS

FIG. 1A is a schematic of a one exemplary embodiment of an injection device of the present disclosure;

FIG. 1B is a schematic of the injection device of FIG. 1A in a deformed state during an injection of fluid into a soft material;

FIG. 2A is a cross sectional view of an exemplary embodiment of a deformable vessel;

FIG. 2B is a cross sectional view of the deformable vessel of FIG. 2A deformed between two rigid bodies;

FIG. 3A is a side view of another exemplary embodiment of an injection device of the present disclosure;

FIG. 3B is a side view of an exemplary embodiment of an injection device with an increased diameter;

FIG. 3C is a side view of an exemplary embodiment of an injection device with an increased diameter;

FIG. 4A is a cross sectional view of another exemplary embodiment of a deformable vessel;

FIG. 4B is a cross sectional schematic of the deformable vessel of FIG. 4A in an un-deformed state;

FIG. 4C is a cross sectional schematic of the deformable vessel of FIG. 4A in a deformed state;

FIG. 5A is a side view of another exemplary embodiment of a deformable conduit comprised of two materials;

FIG. 5B is a side view another exemplary embodiment of a deformable conduit with a depression to receive an actuator;

FIG. 5C is a side view another exemplary embodiment of a deformable conduit with a flexible region and a rigid region;

FIG. 6A is a cross sectional view of another exemplary embodiment of a hexagonal deformable conduit with flexible regions;

FIG. 6B is a cross sectional view of another exemplary embodiment of a deformable vessel with flexible regions;

FIG. 6C is a cross sectional view of the deformable vessel of FIG. 6B in a deformed state;

FIG. 6D is a cross sectional view of another exemplary embodiment of a deformable vessel with a rigid material over molded on a flexible material;

FIG. 7A is a side view of another exemplary embodiment of an injection device;

FIG. 7B is a side cross sectional view of the injection device of FIG. 5A;

FIG. 8 is a side view of exemplary embodiment of an application tool and internal components therein;

FIG. 9 is a diagram of one exemplary hardware system for use in conjunction with the present disclosure;

FIG. 10 is a schematic illustration of one exemplary electronic system for use within the devices of the present disclosure;

FIG. 11A is a side view of an exemplary embodiment of a deformable vessel filled with fluid exhibiting significant micro- and nano-bubbling;

FIG. 11B is a side view of an exemplary embodiment of a thru-filing connection at the input of a vessel;

FIG. 12 is a block diagram of one exemplary embodiment of a computer system for use in conjunction with the present disclosure;

### DETAILED DESCRIPTION

Certain exemplary embodiments will now be described to provide an overall understanding of the principles of the structure, function, manufacture, and use of the devices and methods disclosed herein. One or more examples of these embodiments are illustrated in the accompanying drawings. Those skilled in the art will understand that the systems and methods specifically described herein and illustrated in the accompanying drawings are non-limiting exemplary embodiments and that the scope of the present disclosure is defined solely by the claims. The features illustrated or described in connection with one exemplary embodiment may be combined with the features of other embodiments. Such modifications and variations are intended to be included within the scope of the present disclosure. Terms commonly known to those skilled in the art for components and/or processes of the systems, methods, and compositions disclosed herein and the like may be used interchangeably herein. A person skilled in the art, in view of the claims, present disclosure, and knowledge of the skilled person, will understand such terms are merely examples of such systems, methods, and/or compositions are possible.

To the extent that the present disclosure includes various terms for components and/or processes of the disclosed systems, methods, and the like, one skilled in the art, in view of the claims, present disclosure, and knowledge of the skilled person, will understand such terms are merely examples of such components and/or processes, and other components, designs, processes, and/or actions are possible. By way of non-limiting example, a person skilled in the art will recognize various terms that are used herein interchangeably, such as the terms “vessel,” “tubing,” and “piping” to refer to the tubular conduit within the system that can contain fluid. Further, to the extent features, sides, or steps are described as being “first” or “second,” such numerical ordering is generally arbitrary, and thus such numbering can be interchangeable. Still further, in the present disclosure, like-numbered components of various embodiments generally have similar features when those components are of a similar nature and/or serve a similar purpose. Lastly, the present disclosure includes some illustrations and descriptions that include prototypes or bench models. A person skilled in the art will recognize how to rely upon the present disclosure to integrate the techniques, systems, devices, and methods provided for into a product in view of the present disclosures.

The present disclosure discloses systems, devices, and methods for injecting a micro to nano volume of fluid into a mass of soft tissue to determine mechanical properties of the soft tissue. Without limitation to any one theory or mode of operation, the systems and methods disclosed herein include implementation of volume-controlled cavity expansion (VCCE) to determine mechanical properties of the soft material. Example implementation of VCCE can be described by the relationship between a volume of a fluid introduced into a soft material and the resistance of the material against the formation of a cavity created by the expanding volume of fluid. For soft materials, the expansion of the volume of fluid within the soft material can be dictated by a non-linear relationship between pressure and a local expansion ratio of the volume of fluid. This non-linear pressure relationship encompasses material phenomena such as elastic expansion, fracture progression, viscoelasticity, and more. In other words, the pressure at the cavity wall as the volume of the fluid changes is correlated to material resistance and portions of the non-linear pressure response may be attributed to the elastic modulus of the material, toughness of the material, viscoelastic response of the material, fracture progression of the material, and more.

When a deformable conduit, such as conduit 110, is filled with an incompressible fluid and symmetrically compressed on its radius, nearly all of the linear motion D imposed on the sidewall by the actuator 150 translates to fluid flow, which forms a 12 at the outlet 120. With continued compression over a period of time, the fluid body 12 will symmetrically expand at the outlet 120 until the fluid disperses throughout the material 10.

As a volume of fluid is introduced and expanded, cohesive forces in the fluid and resistance from the soft material initially form the volume into a body of fluid until the cohesive forces and material resistance are overcome and the fluid disperses into the soft material. In an opaque material such as skin or tissue, the cavity expansion and geometry of the fluid body cannot be easily visually measured or characterized and thus, an effective radius can be used to define the cavity expansion. The fluid body can be assumed to expand symmetrically in a spherical shape. The effective radius of the cavity can then be determined from the volume of the fluid body. Precisely knowing the volume of the fluid body at every instance in time enables adequate determination of the effective radius and the effective expansion of the cavity during expansion of the fluid body.

Examples of the present disclosure include the use of a material model corresponding to the tissue or material applied to pressure and cavity expansion relationship to account for the material response to the cavity expansion. For example, a derived result from the neo-Hookean material model can be used to define the elastic modulus as:

$$\frac{p}{E} = \frac{5}{6} - \frac{2}{3 λ} - \frac{1}{6 {λ}^{4}} \qquad Equation 1$$

In Equation 1, E is the elastic modulus, p is the internal pressure, and λ is the circumferential stretch at the cavity wall. Alternative material models such as Ogden or Fung may be used, but use of a material model is not explicitly required to highlight variations in material response for different tissue types. Previous work shows that the results obtained from VCCE methods described adhere well to theoretical predictions and the need for visual characterization of the cavity is eliminated allowing the method to be used to measure material properties under the skin or in opaque tissue.

The relationship between pressure, volume of injected fluid, and the elastic modulus of the material can be influenced by properties such as viscosity, surface energy, and environmental conditions. The systems and methods disclosed herein are designed to account for these properties to precisely introduce a known volume of fluid into the material while measuring the pressure over an amount of time referred to herein as an operation period.

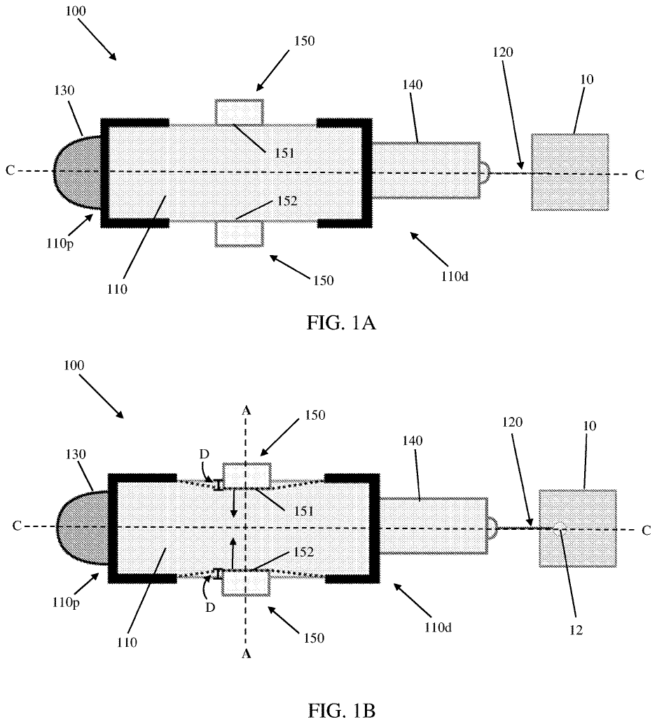

FIGS. 1A and 1B illustrate an exemplary non-limiting embodiment of an injection device 100 capable of utilizing volume control to determine properties of a soft material 10. To obtain accurate measurements from soft tissue, the injection device 100 includes a tubular conduit 110 that can achieve precise displacement of a fluid into soft tissue 10 while measuring the pressure resulting from resistance of the soft tissue 10 as the volume of fluid 12 expands therein. The tubular conduit 110 has a deformable sidewall defining a central channel extending from an inlet 130 at the proximal end 110p to an outlet 120 at the distal end 110p that can contain a fluid therein. The deformable sidewall can deflect by action of an actuator system 150. For example, the tubular conduit 110 can be positioned between two flat plates or surfaces, 151, 152 such that the surfaces 151, 152 contact opposing sides of the tubular conduit 110. The surfaces 151, 152 are aligned parallel to each other and adapted for axial motion to compress the tubular conduit 110. One or both of the surfaces 151, 152 can move a distance D by way of electrical or mechanical means to deflect the sidewall towards the central longitudinal axis of the conduit 110 as shown in FIG. 1B. The plates 151, 152 are positioned transverse to the conduit such that they symmetrically compress or pinch the conduit along the axis A-A perpendicular to the central longitudinal axis C-C of the conduit 110.

When a deformable conduit, such as conduit 110, is filled with an incompressible fluid and symmetrically compressed on its radius, nearly all of the linear motion D imposed on the sidewall by the actuator 150 translates to fluid flow, which forms a 12 at the outlet 120. With continued compression over a period of time, the fluid body 12 will symmetrically expand at the outlet 120 until the fluid disperses throughout the material 10. As the material 10 resists the expansion of the fluid body 12, the pressure within the channel of the conduit can be recorded by a pressure sensor 140 coupled to the conduit 110 until the material 10 relaxes when the fluid body 12 disperses. The time from when deflection of the sidewall is initiated to at least when the measure of pressure (using pressure sensor 140) is sufficiently recorded is herein referred to as the operational period. A sufficient recording of pressure can be acquired from the time when deflection of the sidewall is initiated to when the fluid body 12 disperses. This can operational period can be at least about 0.01 seconds. Depending on the material properties of interest, the operational period can extend up until viscoelastic equilibrium is achieved, which can be up to about 1000 seconds for biological materials. Pressure losses within the device 100 can be accounted for based on the location of the pressure sensor 140 relative to the outlet 120 to determine the pressure of the material 10 at the outlet 120. Pressure losses within the device 100 can be minimized by reducing the distance of the pressure sensor 140 to the outlet 120. The resisting pressure of the material at the outlet 120 can be compared against volume to create a pressure-volume curve from which numerous mechanical properties of the material 10 can be determined or observed, such as the elastic modulus.

To determine elastic modulus of the material 10 using VCCE, the volume of the cavity created by the fluid body 12 should be known at every time instance during expansion of the fluid body 12 to determine the effective expansion of the cavity. To accomplish this, the tubular conduit 110 is designed and modeled to accurately introduce a known volume of fluid at a controlled radial expansion rate as a result of sidewall deflection during the operation period. The known volume of fluid can be on the micro or nanoliter scale. For example, the total volume of fluid displaced from the central channel and introduced into the soft material during operation can be between 1 nanoliter and 1000 nanoliters or between 1 microliter and 1000 microliters. These displacement ranges are representative and non-limiting. In some embodiments, the fluid is introduced into the material at a constant rate of 16 mm/s from a volume of about 0 microliters to about 2.10 microliters.

Notably, while experimentally determining a volume of fluid dispensed through deformable conduits using standard methods might be feasible with incompressible fluids in ideal, incompressible tubular conduits, real-world applications seldom align with these conditions. Traditional tubular conduits often exhibit production inconsistencies, which reduces the accuracy and repeatability of any experimental result. Further, when experimentally evaluating a tubular conduit to ascertain precise volumes of fluid in polymers, even the highly controlled production of polymers exhibit non-trivial heterogeneity, even for systems that can normally be considered as homogeneous. This variability compromises both the accuracy and precision of volume measurements across multiple experimental trials.

Furthermore, imaging techniques, which could potentially be employed to estimate fluid volumes, face limitations with opaque materials. Even when transparent polymers are used, their inherent heterogeneity can disrupt the processing needed for precise volume measurements due to unpredictable refractive properties. Minimizing the role of refraction along with reducing material inconsistencies to acceptable levels remains challenging and creates severe limitations in producing a desired output for volume of fluid dispensed.

Moreover, accurately determining a three-dimensional volume of dispensed fluid requires the use of multiple cameras simultaneously, as fluids introduced into soft materials do not form perfect spheres. Although, in diagnostics, it can be assumed that the volume of fluid introduced into the system is an effective sphere, and it closely resembles a sphere in practice, this is an effective assumption, with exact control over the volume of fluid introduced into the material, as provided by aspects of the present disclosure. Without knowing the exact volume of fluid introduced into the material, an exact measure of the fluid volume dispensed must be conducted first to then once again correlate that volume to an effective sphere for diagnostic utility. Further, the dynamic behavior of soft materials, which is an actively researched area, further complicates measurements, as a measured volume dispensed in a material is time-dependent and thus even further difficult to resolve.

In addition, existing ultrasound technology also does not provide the resolution needed to measure the small volumes of fluid (e.g., nanoliters) involved in critical phenomena prior to material fracture.

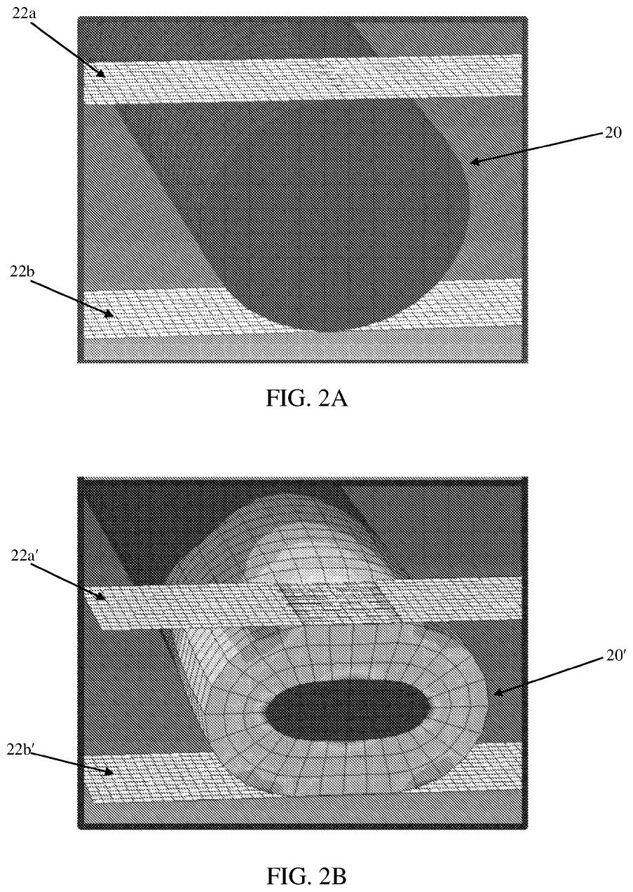

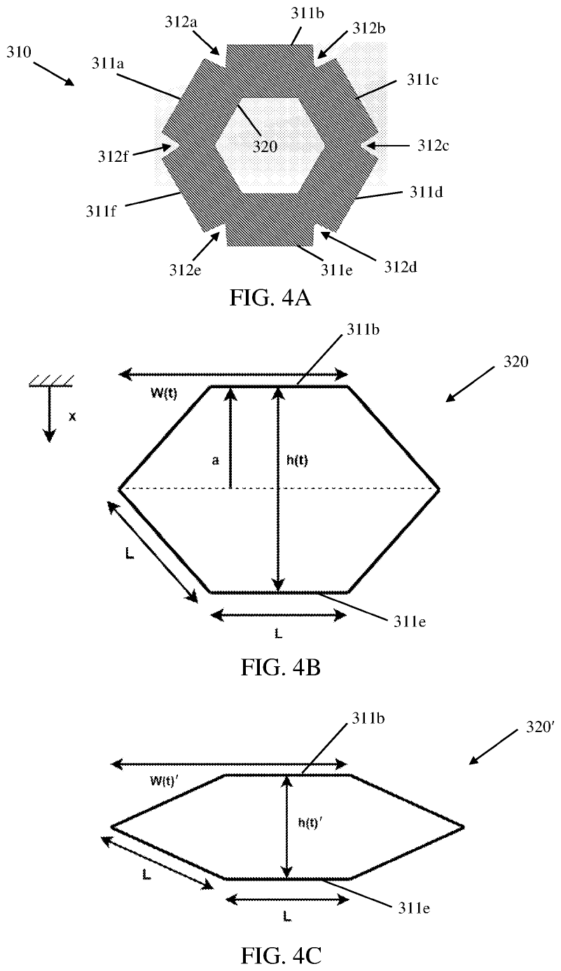

Examples of the present disclosure include deformable conduits whose geometry is modeled to determine the exact relationship between the increasing distance D of sidewall displacement during the operation period and the changing volume of fluid injected during the operation period. One example of a conduit geometry is a deformable cylinder 20. The deformation profile of a cylinder conduit 20 is shown in FIGS. 2A and 2B. FIG. 2A shows a cross section of an un-deformed cylinder 20 positioned between two rigid surfaces 22a, 22b of an actuator. FIG. 2B shows a cross section of the cylinder 20′ as the distance between rigid bodies 22a′, 22b′ reduced to symmetrically deform the cylinder 20′. The non-linear deformation profile illustrated can be used to determine distance the actuator needs to move to dispense the desired volume of fluid over the operation period. The relationship between actuator surface position and volume of displaced fluid over time for the cylindrical conduit 20 is provided in equation 2:

$${\dot{x}}_{p} = {\underbrace{[ 4 π {( \frac{d {V}_{g} ( {x}_{p} )}{d {x}_{p}} )}^{- 1} {( \frac{4 π}{3} {V}_{9} ( {x}_{p} ) )}^{\frac{3}{2}} ]}}_{F ( {x}_{p} )} {\dot{r}}_{g} \qquad Equation 2$$

wherein xₚ represents the position of the actuator, V_{g}(xₚ) represents the growth of the fluid body at the outlet, \dot{r}_{g} represents a constant radial growth rate of the fluid body, and \dot{x}ₚ represents the rate of actuator motion. This predetermined relationship between actuator distance and the volume of fluid dispensed can be embedded into a controller configured to drive the actuator 150 to displace the sidewall axially at the distance D required to dispense the desired volume of fluid from the outlet at any predetermined radial expansion rate. Other conduit geometries can be analyzed similarly to the cylinder 20, 20′ to precisely determine the relationship between sidewall deflection distance and fluid displacement as will be discussed with respect to FIG. 4A

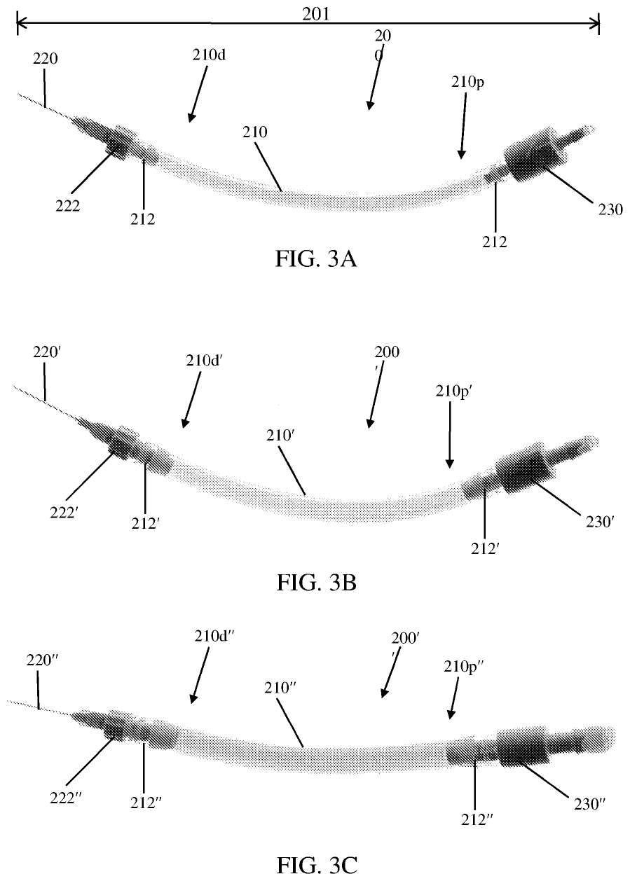

FIG. 3A illustrates an exemplary non-limiting embodiment of an injection device 200 with a cylindrical conduit 210. The conduit 210 has a deformable sidewall extending from an inlet 230 at the proximal end 210p to an outlet 220 at the distal end 210p defining a channel that contains an incompressible fluid therein. In the illustrated embodiment, the outlet is a syringe needle 220 fluidly coupled to the distal end of the vessel 210d. The needle 220 size and length can be chosen based on the type, location, and depth of the tissue being tested. In some embodiments, the outlet can be inserted into the soft tissue at any depth between below the skin surface; however, the relationship of boundary effects between the soft tissue and fluid should be known. For this, we continue with the aforementioned neo-Hookean material model and elucidate the circumferential stretch at the boundary 2b:

$${λ}_{b} = {\frac{b}{B} [ ( 1 + ( {λ}^{3} - 1 ) ) {( \frac{A}{B} )}^{3} ]}^{1 / 3} \qquad Equation 3$$

$$\frac{p}{E} = \frac{1}{6} [ ( {λ}_{b}^{- 4} + 4 {λ}_{b}^{- 1} - {λ}^{- 4} - 4 {λ}^{- 1} ) \qquad Equation 4$$

The cavity is defined as an effective sphere with initial defect, A, that expands to effective radius, a, in a soft material of undeformed radius, B, which under the influence of pressure, p, deforms to effective radius, b. The ratio B/A captures the relationship between the size of the boundary and the initial defect size.

One with skill in the art will appreciate that the device 200 can have varying outlet 220 lengths to reach deep tissue. In the present example, the syringe needle is 25 gauge needle with a length of approximately one to two inches. A one-way valve 230 can be disposed at the inlet and configured to allow fluid to flow into the vessel while preventing fluid from flowing out of the vessel. Fluid tight seals 212 are disposed at the distal 210d and proximal 210p ends of the vessel. The seals 212 can be compatible with luer fittings. For example, the needle 220 can be coupled to the distal end of the vessel through a luer lock 222 to create a fluid tight seal between the vessel and the needle 220.

The length 201 of the conduit 210 is substantially longer than the internal diameter to ensure laminar fluid flow through the conduit 210. More specifically, the ratio of conduit length 201 to the inner radius of the conduit 210 can be greater than the Reynolds number of the fluid divided by 48. For example, the conduit 210 in FIG. 3A has an inner diameter of approximately 0.125 inches and a length of approximately 4 inches. The conduit 210′ in FIG. 3B has an inner diameter of approximately 0.1875 inches and a length of approximately 4 inches and the conduit 210″ in FIG. 3C has an inner diameter of approximately 0.250 inches and a length of approximately 4 inches. The geometry of the conduits can be configured to ensure that fluid flow is sufficiently laminar.

The deformable wall of the conduit 210 can be an elastic material capable of deflection while maintaining enough rigidity to exhibit negligible expansion when pressure within the range of the materials tested is applied. For example, the deformable wall can be a flexible polymer with a Young's modulus of about 6e⁷ Pa and Poisson's ratio of 0.45 that does not significantly expand under about 25 kPa of pressure. Most materials undergo volumetric expansion with fluid fracture occurring approximately in the range of about 0 nanoliters to about 1000 nanoliters or approximately in the range of about 1 nanoliter to about 1000 nanoliters. Thus, it can be assumed that fluid fracture occurs at the highest recorded pressure of the system and as such, compression of the conduit must be negligible with respect to conduit deformation at expected peak pressures corresponding to fluid fracture. The material properties of the vessel wall and the design of the vessel result in a configuration with a negligible expansion quality factor defined as the degree of tube expansion under pressure. Materials with differing elastic properties can be used for higher-pressure conditions above 25 kPa. In some embodiments, the vessel material exhibits negligible expansion under between about 1 kPa and 500 kPa of pressure.

The deformable conduits disclosed herein can have alternative geometries such as a hexagon geometry, the cross-section of which is illustrated in FIGS. 4A-4C. Similar to the previously described embodiments, the relationship between deflection of the conduit and the volume of the fluid dispensed from the outlet is predetermined such that a controller associated with the device can move an actuator to deflect the conduit the required distance to dispense a desired volume of fluid at the micro or nano liter scale. As shown in FIG. 4A, a hexagonal conduit 310 can comprise six sidewalls 311a-f joined by flexible hinged regions 312a-f such that each sidewall 311a, 311b, and 311c is parallel to an opposing sidewall 311d, 311e, and 311f, respectively, around an inner circumference 320. Each of the flexible hinged regions 312a-f are capable of bending to change angle and allow the conduit 310 to deform. Wedges can be removed from the external surface of the sidewall to create the flexible regions 312a-f and a continuous inner surface 320. Similar to the embodiments described above, the hexagonal conduit 310 can be compressed between two flat parallel surfaces of an actuator system. For example, a first flat surface can be in planar contact with a sidewall 311b and the second flat surface can be in planar contact with the opposing sidewall 311e. One or both of the flat surfaces can move axially in the direction x towards the central longitudinal axis of the conduit as shown in FIG. 4C.

FIG. 4B shows the inner surface 320 of the hexagonal conduit 310 in an initial un-deformed configuration having an initial height h, an initial width w and a sidewall length L. Linear motion of the actuator surfaces moves the sidewalls 311b, 311e towards each other and the angles at the flexible joint regions change as a result thereby compressing the height h′ and expanding the width w′ as shown in FIG. 4C. As the conduit 310 is compressed, the sidewall length L and inner surface 320 perimeter, remain unchanged. The height and width of a cross-section of the hexagonal conduit 310 as functions of time are shown in equations 5 and 6:

$$h ( t ) = 2 ( \frac{L \sqrt{3}}{2} ) + {h}^{′} ( t ) t + \frac{1}{2} {h}^{″} ( t ) {t}^{2} \qquad Equation 5$$

$$W ( t ) = L + \sqrt{{L}^{2} - {( \frac{L \sqrt{3}}{2} + \frac{{h}^{′} ( t ) t}{2} + \frac{{h}^{″} ( t ) {t}^{2}}{4} )}^{2}} \qquad Equation 6$$

From equations 3 and 4, the cross-sectional area Aₓ(t) of the hexagonal conduit at the portion of deflection can be determined at all instances of time within the operation period. The height h(t), width w(t), and area Aₓ(t) curves can be modified for actuator movement a constant velocity h′(t) and constant acceleration h″(t). Further, by applying conservation of mass to the system, the relationship between the cross sectional area change Aₓ(t) and the volumetric flow rate Qₛ(t) at the outlet can be determined as shown in equation 7:

$${V}_{x} ( t ) {A}_{x} ( T ) = {V}_{s} ( t ) {A}_{s} = {Q}_{s} ( t ) \qquad Equation 7$$

With a known and constant conduit length, the internal volume of the conduit 310 can be determined at all instances of time and, desirably, the volumetric rate of fluid displacement from the conduit 310 can be precisely controlled over an operation period. The relationship between volumetric flow rate and conduit cross sectional area from equation 7 can be used with the deflection profiles from equations 5 and 6 to determine a distance profile of actuator movement required to dispense a desired volume of fluid. As discussed with respect to FIG. 2, the predetermined relationship between the deformation of the hexagonal vessel and the volume of fluid injected can be determined and embedded into the system to control the linear movement of the flat plates.

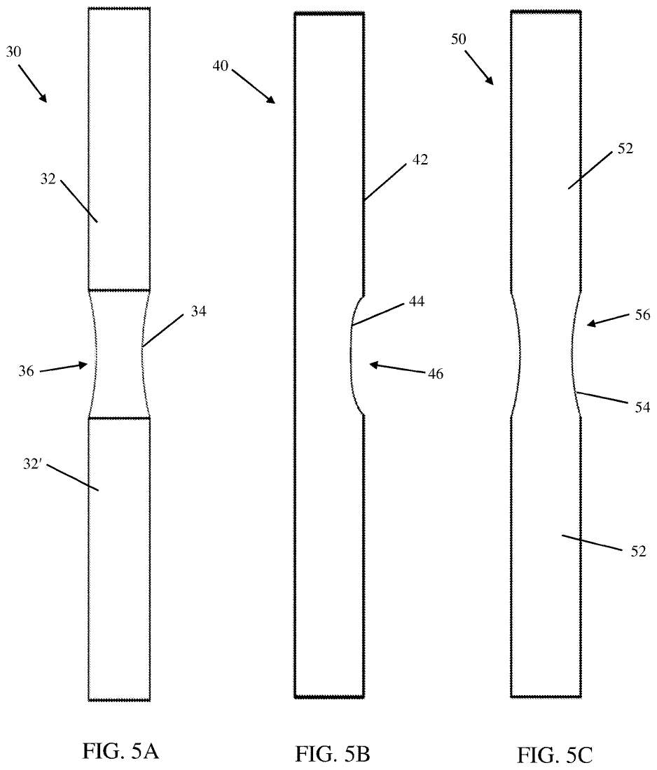

Additional examples of conduit configurations are illustrated in FIGS. 5A-5C. FIG. 5A shows a conduit 30 comprised entirely of a rigid material 32 with an exposed region 36 comprised of a flexible material 34 configured to deform under pressure from the actuator. Some conduits 40 can be shaped to contain a dimple 46 or depressed region on the sidewall configured to receive the actuator. In these examples, the dimple 46 can be shaped into a conduit comprised of a single flexible material, or alternatively, the dimple 46 can be comprised of a flexible material 44 while the remaining conduit 40 sidewall is comprised of a rigid material 42. FIG. 5C illustrates a conduit 50 with a symmetric depressed region 56 around the circumference of the conduit 50 to receive a first and a second flat plate of actuator symmetrically.

In other examples, the conduit can comprise four or more rigid sidewalls with flexible regions therebetween to enable symmetric deformation as described with respect to FIG. 4A. In these examples, at least two flexible regions are needed to enable symmetric deformation. The flexible regions can be extruded from the rigid material to create regions with less thickness to enable deformation. The flexible regions of any embodiment of the present devices can be made from a flexible polymer material or metal capable of deforming under actuator pressure.

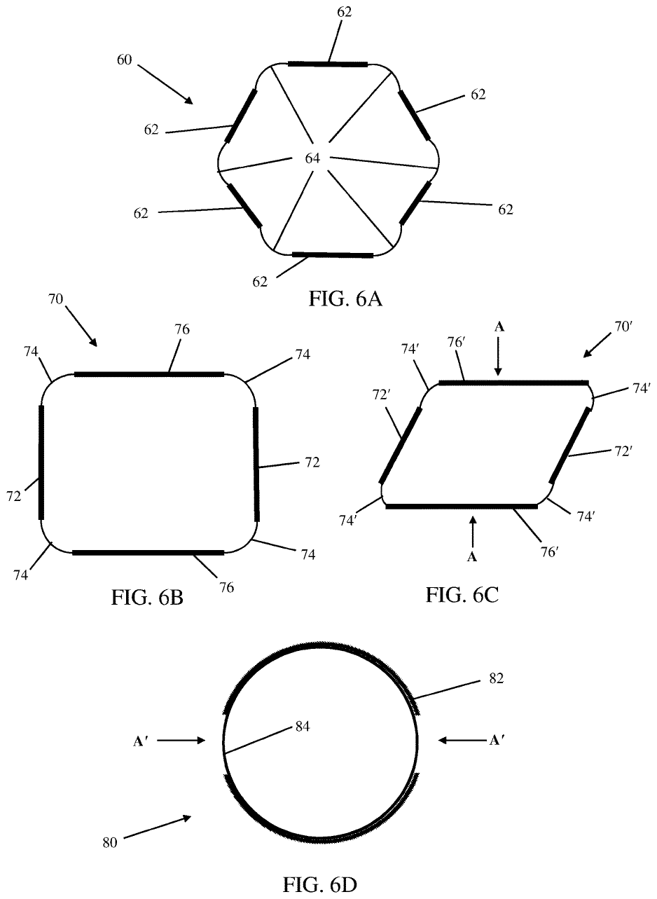

The cross sections of additional conduit configurations are illustrated in FIGS. 6A-6D. FIG. 6A shows a hexagonal conduit 60 with six sidewalls 62 comprised of a rigid material. A flexible material 64 can be disposed between the sidewalls 62 to arrange the sidewalls 62 in a convex hexagonal arrangement. The flexible material 64 enables the conduit to deflect under compression while maintaining a constant perimeter. In other examples, the conduit can comprise four or more rigid sidewalls with flexible regions therebetween to enable symmetric deformation as described with respect to FIG. 4A. Similar to the hexagonal geometry, the rectangular geometry comprises two primary sidewalls 76, that are parallel to each other. Secondary sidewalls 72 are disposed between the primary sidewalls 76. The sidewalls 76, 72 can be comprised of a rigid material. Flexible regions can be formed between sidewalls 76, 72 by removing rigid material to create thinner regions as previously described or a flexible material 74 can be disposed between the rigid sidewalls 72, 76. Force can be applied to the primary sidewalls 76′ in the direction A to deform the conduit as shown in FIG. 6C.

In some examples, a rigid material 82 can be over molded on a conduit 80 comprised of a flexible material 84 as shown in FIG. 6D. The rigid material is over molded to expose regions of the flexible material 84 configured to receive force from an actuator in the direction A′. In this example, the flexible material 84 lines the interior channel of the conduit 80. In some embodiments, the flexible material 84 can cover the exterior surface of the rigid material 82 over the exposed regions. The flexible regions disclosed in any of FIGS. 4A-6D can be extruded from rigid material to create regions with less thickness to enable deformation or can be made from a flexible polymer material or metal capable of deforming under actuator pressure.

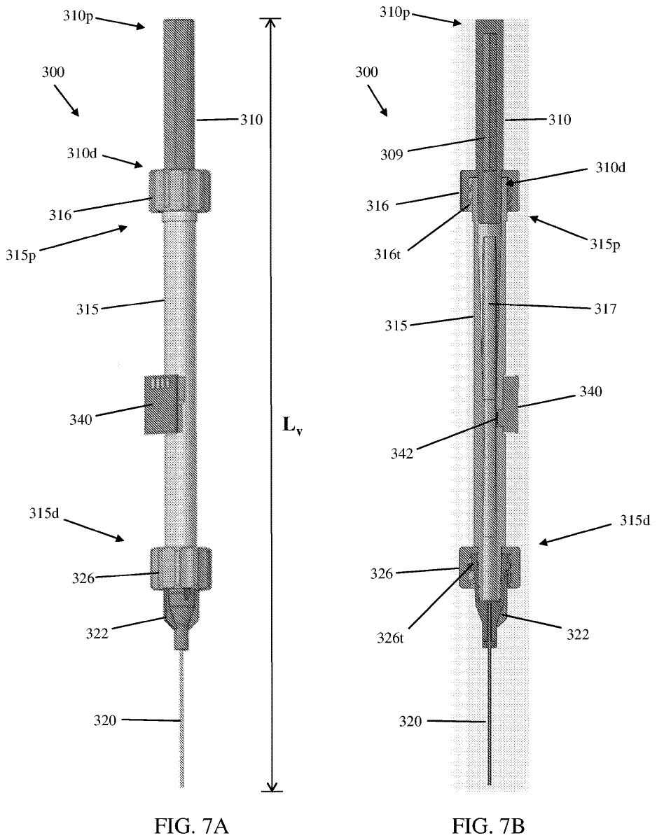

An injection device 300 utilizing a hexagonal conduit 310 is further illustrated in FIGS. 7A and 7B. The deformable conduit 310 can be coupled to a rigid conduit 315 such that the channel 309 of the deformable conduit 310 is in fluid communication with the channel 317 of the rigid conduit. The channels 315, 317 of the conduits 310, 315 can have the same internal diameter to create consistent fluid flow conditions through the device 300. The deformable conduit 310 and rigid conduit 315 can be connected through fluid tight seal such as a luer lock. A cap 316 can be disposed around the distal end 310d of the deformable conduit 310. Internal threading within the cap 316 corresponds to at least one thread 316t on an outer portion of the proximal end 315p of the rigid conduit and configured to secure the deformable conduit 310 to the rigid conduit 315. Similarly, a threaded cap 326 can be disposed on the distal end 315d of the rigid conduit 315 to mate with at least one thread 326t on a needle cap 322 to secure the needle 320 to the rigid body 315. One with skill in the art will recognize that other means to join the components of the injection device 300 to create a fluid tight seal between the components are possible.

The compression of the conduit can be accomplished by displacing a single plate against the conduit while the opposing plate is stationary or each of the plates can compress against the conduit simultaneously. As discussed with respect to FIG. 1B, the flat plates can symmetrically compress the conduit at a radius along the length of the conduit. The plates can have any length, for example such that the rate of fluid injection is tunable to the plate profile, size, and geometry. For example, the plate can be less than 10% of the length of the conduit so that the conduit wall is deformed at only a single point or portion of the conduit wall. Deformation at a single point or portion may be beneficial to control the rate of injection. In other embodiments, the plates can be substantially the same length or longer than the deformable portion of the conduit and the compression can occur over the entire length of a deformable portion which can be beneficial in situations where faster injection rates or larger injected volumes are preferred.

In the illustrated embodiment 300, a pressure sensor 340 is disposed on the rigid conduit 315. A portion of the pressure sensor 342 can extend through the conduit 315 sidewall such that it can record the pressure within the channel 317. In some embodiments, a diaphragmatic pressure sensor can be disposed in line with conduit between the outlet 320 and the proximal end of the deformable conduit 310p. To minimize the effect of pressure loss in the system, the pressure sensor 340 can be disposed closer to the outlet 320 than to the proximal end 310p. Other pressure sensors known in the art such as optical or strain based pressure sensors can be used. For example, a piezoresistive pressure sensor can be incorporated into the device. Pressure loss can occur in the system between the pressure transducer 340 and the outlet 320. To account for potential minor pressure losses due to head losses and viscous effects, conservation of momentum laws can be applied to determine the system's theoretical pressure distribution. The total pressure loss can be factored into the measurements from the pressure sensor 340 to determine an accurate pressure at the outlet 320 within the soft material.

The tube geometry discussed above can be embedded within the system such that data is read and the time required to perform the VCCE protocol is translated to the necessary number of steps required for the stepper motor to carry out the operation. Additionally, conservation of momentum laws or other models can be embedded to enable the system to account for pressure loss when determining the elastic modulus using the measurements from the pressure sensor.

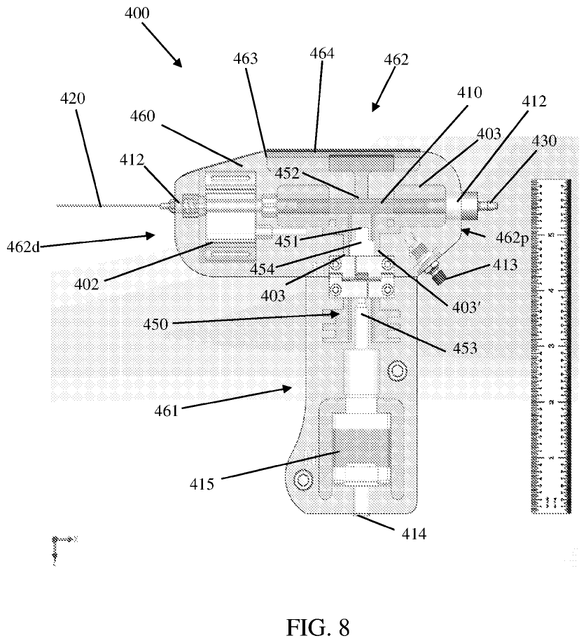

Examples of the deformable injection devices disclosed herein can be incorporated in a handheld injection system 400, as illustrated, for example, in FIG. 8. The handheld injection system 400 includes a housing 460 with a handle portion 461 and a body portion 462 defining an inner cavity to house injection devices, such as injection devices 100, 200, 300, and 410 and associated components. Due to the single-use nature of the injection device 410, a spring-loaded top cover 464 can be disposed over an opening 463 formed within the body portion 462 of the housing 460 to access components within and replace the disposable injection device 410. In the example illustrated, an injection device 410 is positioned within the body portion 462 such that the inlet 430 extends from an opening formed in the proximal end 462p of the housing body and the outlet 420 extends from an opening formed in the distal end 462d of the housing body. In some embodiments, luer-barb compatible fittings 412 can be disposed at the housing openings to create a seal with the distal and proximal ends of the injection device 410. Fixation and support structures 402, 403 can be 3D printed or otherwise disposed within the housing 460 to secure the injection device 410, actuator 450, and other electrical components within the housing 460. In FIG. 8, a clip 402 is used to removably secure the injection device 410 within the housing 450.

The linear actuator system 450 of the handheld injection system 400 includes a shaft 453 associated with a first flat surface 451. The first flat surface 451 can be disposed on a drive block 454 disposed between rails 403, 403′. The drive block can be coupled to the rails 403, 403′ such that all movement other than vertical movement along the rails 403, 403′ is restricted. Vertical movement of the shaft 453 is driven by a motor 415. As the shaft 453 moves vertically, it drives the drive block 454 and first surface 451 towards the injection device 410 while the rails 403, 403′ ensure linear movement of the drive block 454 perpendicular to a central longitudinal axis of the injection device 410. The actuator 450 is disposed within the housing 460 such that the first flat surface 451 is disposed proximal to the injection device 410. A second flat surface 452 is fixed proximal to the injection device 410 opposing the first flat surface 451. A battery (not shown) can be included within the housing 460 to power the motor 415 such that the shaft 453 extends and moves the first flat surface 451 towards the second flat surface 452 compressing the injection device 410 between the surfaces 451, 452. Actuator mechanisms used with the disclosed devices are capable of micro- to nano-meter stepping. In some examples, the actuator system 450 can attain a 3.2 micrometer step resolution. One skilled in the art will appreciate that the step resolution will depend on particular form-factors and any included transmission arrangements, which may introduce space-constraints in hand-held devices. In many embodiments, motors and mechanisms capable of micro- to nano-meter stepping are of sufficient resolution. In other examples, the actuator system 450 can have a millimeter step resolution and additional motors, actuators, or other transmission systems such as wedges or levers can be further implemented to improve the step resolution. Although the illustrated actuator 450 compresses the injection device 410 by movement of a single plate 451 towards a stationary plate 452, each plate 451, 452 can be disposed on a shaft 453 or drive block 454 and configured to move simultaneously towards each other to compress the injection device 410 in between.

In some embodiments, the actuator system 450 can further comprise a second pressure sensor disposed proximal to the first flat surface such that the second pressure sensor can measure the pressure of the first flat surface against the injection device 410. The second pressure sensor can relay this pressure measurement to the controller and/or processor to provide in-situ feedback of actuator displacement and deformation of a conduit. Alternatively, the second pressure sensor can relay the pressure measurement to the user through the controller and/or processor and a display associated with the processor for manual control of the actuator. The second pressure sensor can further provide a baseline for actuator positioning. For example, the initial position of the actuator against the conduit wall can be based on a specific or zero pressure measurement from the second pressure sensor and the displacement can occur based on an increase in pressure recorded from the second pressure sensor.

Additionally, other power sources can be used with the system 400 and as such the housing 460 can include connections 413, 414 to electrically couple the system 400 with a power source or a processor. Although a portable handheld system 400 is illustrated as an example, injection systems and devices of the present disclosure can be incorporated into a larger benchtop housing.

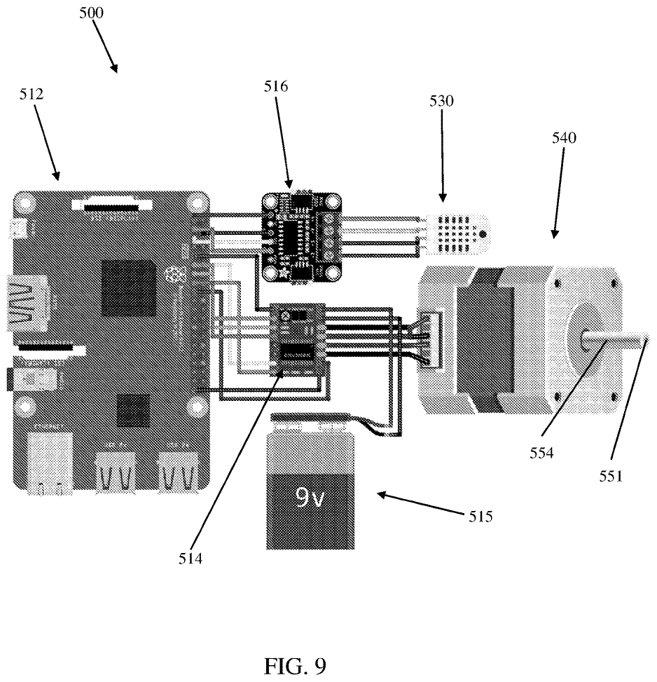

FIG. 9 illustrates an exemplary system 500 of electrical components that can be included in the housing 460 along with the injection device 410. The system 500 can include a processor 512 configured to simultaneously interface with a motor controller 514 and receive pressure measurements from a pressure sensor 530 through an Analog to Digital Converter (ADC) 516. Increased resolution of the motor 540 can be achieved through coupling mechanisms between the motor 540 and a first surface 541 such as the motor driver 454 between rails 403, 403′ as described with respect to FIG. 8. A battery 515 can be included to serve as a power source for the motor controller 540 or processor 512.

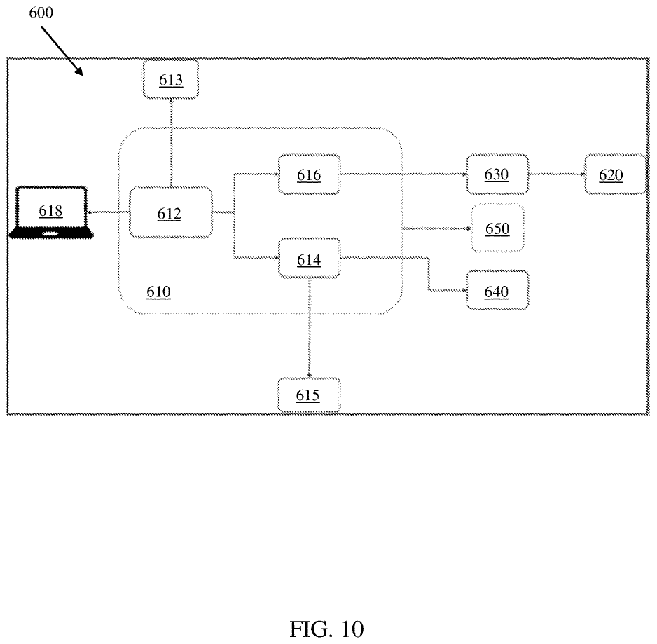

FIG. 10 illustrates diagram of another exemplary system 600 used in conjunction with the disclosed injection devices. In this example, a central circuit board 610 that can fit within the handheld housing 460 contains a processor 612, a motor 614, and ADC 616. A deformable conduit 620 is coupled with a pressure sensor 630 as previously described and the pressure sensor 630 is operatively connected to the ADC 616, which is configured to receive and process signals from the pressure sensor 630 and send those signals to the processor 612. A power source, such as a battery 615, can power the motor 614. The processor 612 can alternatively be powered through an electrical outlet 613. In some embodiments, the system include can include switch 650 configured to initiate the actuator to inject fluid and begin collecting the pressure measurements during injection. As discussed, the injection devices and systems can be configured with a predetermined relationship between conduit sidewall deflection and the volume of fluid injected. The deformation model can be embedded or stored in the processor 612 such that the processor 612 can determine the time required to dispense a known volume of fluid and translates the required time into a displacement distance profile. The processor 612 then instructs the motor 614 to carry out a required amount of steps over the amount of time. A second actuator 640 can be placed between the motor 614 and proximal to the conduit 620 to increase the motor 614 resolution and achieve the required displacement distance profile to inject the known volume of fluid. In some embodiments, the processor 612 is coupled to a display 618 and configured to visually display the pressure-volume response of the soft material or other visual data such as sidewall deflection and volume curves, pressure measurements, pressure and cavity expansion curves, and calculated material properties thereon.

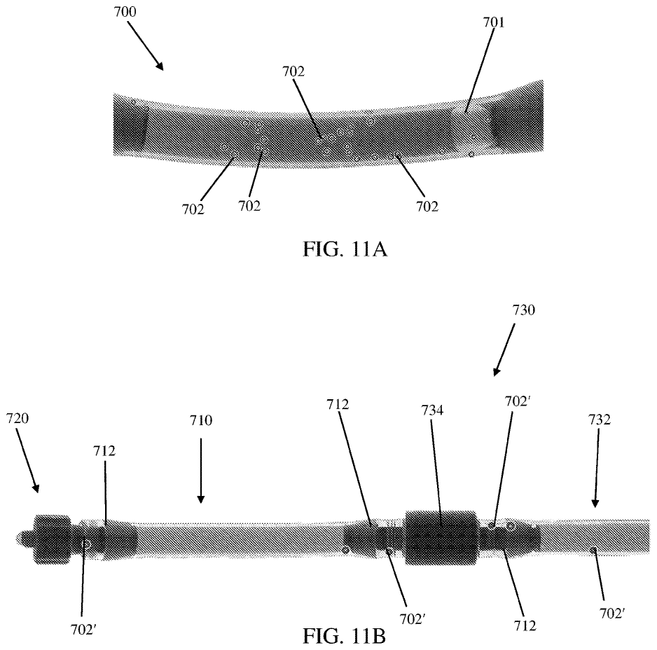

Air bubbles introduced into the channel of the conduit with the fluid can add unwanted compressibility to the system and skew results. Examples of the present disclosure include through-filling methods that can be used in conjunction with the devices and systems of the present disclosure to fill a tubular conduit while ensuring that unwanted air bubbles are not passed into the internal volume of the conduit. FIG. 11A illustrates a tubular conduit 700 filled with a fluid including a large visible air bubble 701. In addition to visible air bubbles 701, microbubbles 702 can be present throughout the fluid introducing system compressibility. FIG. 11B illustrates an embodiment of a tubular conduit 710 with a through-filling mechanism 730 at the inlet. The through-filling mechanism 730 comprises a fill tube 732 coupled to the proximal end of the conduit 710p through a fluid tight seal 712. A one-way valve 734 can be disposed between the fill tube 732 and the conduit 710 such that fluid can flow from the fill tube 732 into the conduit 710 but fluid flow out of the conduit 710 is restricted. To fill the conduit 710 with fluid devoid of air, the fill tube 732 is filled with fluid and the fluid is allowed to flow slowly into the previously empty conduit 710. Due to the viscous movement of the fluid from the fill tube 732 to the conduit 710, a ‘no-flow’ condition occurs at the wall of the fill tube 732 trapping air 702′ against the wall of the fill tube 752. Through-filling the conduit 710 as described eliminates the need for vacuum air removal or physical agitation to remove air bubbles while ensuring the system is relatively free of visible and micro air bubbles. A through-filling method can fill the conduit 710 with fluid and ensure that any compressibility added to the system by air in the fluid is negligible. Microbubbles 702, 702′ can be identified in the fluid through image processing software. Image processing shows that the described through-filling method can eliminates nearly all of the microbubbles as can be seen by comparing FIG. 11B to FIG. 11A.

### Diagnostic Method Examples

In medical environments, there is a critical need for tissue health monitoring and diagnostics at the point-of-care. Examples of the present disclosure include systems and devices configured to interface seamlessly with both specialists and non-specialists, requiring minimal training. Example systems and devices can operate by utilizing a flexible conduit, which may be pre-filled with an incompressible fluid such as saline either off-site at a manufacturing facility or on-site at a practitioner's or specialist's place of work. This conduit is then loaded into either a handheld or a benchtop-equivalent device. Upon loading, the device is prepared for diagnostic testing.

In operation, a needle of the device is inserted into the target tissue of the patient. Following insertion, an actuation mechanism precisely deforms the conduit, causing the displacement of a controlled volume of fluid, ranging from nano- to micro-liters, into the tissue. Concurrently, the device records the pressure exerted during the injection. Once the fluid volume is no longer controlled and all relevant material physics characteristics have been documented, the needle is withdrawn from the patient. Pressure can also be recorded during needle insertion and withdrawal. Although, there is no volume of fluid being inserted into the target tissue during needle insertion and withdrawal, the physics of insertion and withdrawal can be captured in the material response via the pressure sensor and may be used as additional data towards determining material parameters and tissue health.

The collected data, which includes metrics such as pressure, volume, and time, can then be processed either locally within the device or externally. This analysis can include determining material properties of the tissue, which can be compared against benchmarks typical of healthy tissue or used to understand disease progression. By quantifying these material properties or providing a comparative analysis of diseased versus healthy tissue, examples of the present disclosure enable significant enhancement of point-of-care diagnostics for healthcare providers and patients.

### Computer System Examples

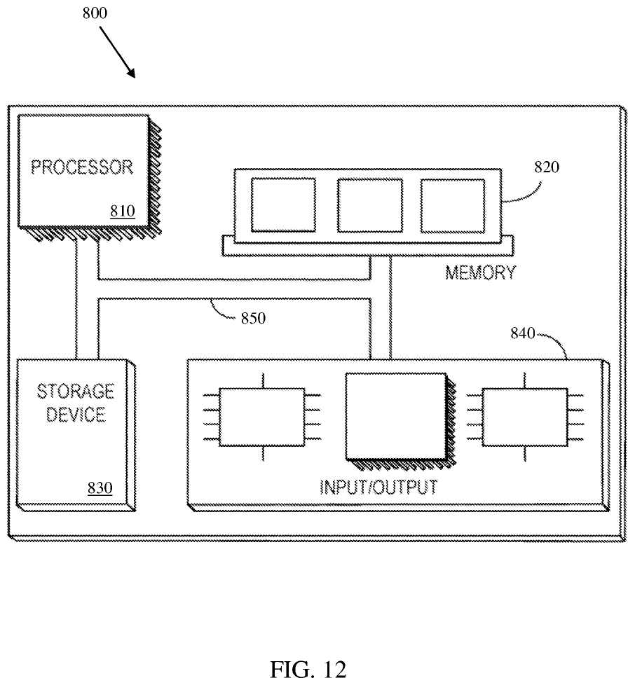

FIG. 12 provides for one non-limiting example of a computer system 800 upon which the present disclosure can be built, performed, trained, etc. For example, referring to FIGS. 7 and 8, the processing modules or processors 512, 612 can be examples of the system 800 described herein. The system 800 can include a processor 810, a memory 820, a storage device 830, and an input/output device 840. Each of the components 810, 820, 830, and 840 can be interconnected, for example, using a system bus 850. The processor 810 can be capable of processing instructions for execution within the system 800. The processor 810 can be a single-threaded processor, a multi-threaded processor, or similar device. The processor 810 can be capable of processing instructions stored in the memory 820 or on the storage device 830. The processor 810 may execute operations such as calculation of a actuator displacement as a function of time based a vessel model, calculation of a pressure-volume response based on information received from a sensor, or adjustment of an effective cavity radius, among other features described in conjunction with the present disclosure, including any control logic associated with the operation of a VCCE injection system or device.

The memory 820 can store information within the system 800. In some implementations, the memory 820 can be a computer-readable medium. The memory 820 can, for example, be a volatile memory unit or a non-volatile memory unit. In some implementations, the memory 820 can store information related to modeled vessel geometries, fluid flow parameters (predicted or measured), soft material properties, and any information related to the calculation of elastic properties, among other information.

The storage device 830 can be capable of providing mass storage for the system 500. In some implementations, the storage device 830 can be a non-transitory computer-readable medium. The storage device 830 can include, for example, a hard disk device, an optical disk device, a solid-date drive, a flash drive, magnetic tape, or some other large capacity storage device. The storage device 830 may alternatively be a cloud storage device, e.g., a logical storage device including multiple physical storage devices distributed on a network and accessed using a network. In some implementations, the information stored on the memory 820 can also or instead be stored on the storage device 830.

The input/output device 840 can provide input/output operations for the system 800. In some implementations, the input/output device 840 can include one or more of network interface devices (e.g., an Ethernet card), a serial communication device (e.g., an RS-232 10 port), and/or a wireless interface device (e.g., a short-range wireless communication device, an 802.11 card, a 3G wireless modem, or a 4G wireless modem). In some implementations, the input/output device 840 can include driver devices configured to receive input data and send output data to other input/output devices, e.g., a keyboard, a printer, and display devices. In some implementations, mobile computing devices, mobile communication devices, and other devices can be used.

In some implementations, the system 800 can be a microcontroller. A microcontroller is a device that contains multiple elements of a computer system in a single electronics package. For example, the single electronics package could contain the processor 810, the memory 820, the storage device 830, and input/output devices 840.

Although an example processing system has been described above, implementations of the subject matter and the functional operations described above can be implemented in other types of digital electronic circuitry, or in computer software, firmware, and/or hardware, including the structures disclosed in this specification and their structural equivalents, or in combinations of one or more of them. Implementations of the subject matter described in this specification can be implemented as one or more computer program products, i.e., one or more modules of computer program instructions encoded on a tangible program carrier, for example a computer-readable medium, for execution by, or to control the operation of, a processing system. The computer readable medium can be a machine-readable storage device, a machine-readable storage substrate, a memory device, a composition of matter effecting a machine-readable propagated signal, or a combination of one or more of them.

Various embodiments of the present disclosure may be implemented at least in part in any conventional computer programming language. For example, some embodiments may be implemented in a procedural programming language (e.g., “C”), or in an object-oriented programming language (e.g., “C++”). Other embodiments of the invention may be implemented as a pre-configured, stand-along hardware element and/or as preprogrammed hardware elements (e.g., application specific integrated circuits, FPGAs, and digital signal processors), or other related components.

The term “computer system” may encompass all apparatus, devices, and machines for processing data, including by way of example a programmable processor, a computer, or multiple processors or computers. A processing system can include, in addition to hardware, code that creates an execution environment for the computer program in question, e.g., code that constitutes processor firmware, a protocol stack, a database management system, an operating system, or a combination of one or more of them.

A computer program (also known as a program, software, software application, script, executable logic, or code) can be written in any form of programming language, including compiled or interpreted languages, or declarative or procedural languages, and it can be deployed in any form, including as a standalone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

Such implementation may include a series of computer instructions fixed either on a tangible, non-transitory medium, such as a computer readable medium. The series of computer instructions can embody all or part of the functionality previously described herein with respect to the system. Computer readable media suitable for storing computer program instructions and data include all forms of non-volatile or volatile memory, media and memory devices, including by way of example semiconductor memory devices, e.g., EPROM, EEPROM, and flash memory devices; magnetic disks, e.g., internal hard disks or removable disks or magnetic tapes; magneto optical disks; and CD-ROM and DVD-ROM disks. The processor and the memory can be supplemented by, or incorporated in, special purpose logic circuitry. The components of the system can be interconnected by any form or medium of digital data communication, e.g., a communication network. Examples of communication networks include a local area network (“LAN”) and a wide area network (“WAN”), e.g., the Internet.

Those skilled in the art should appreciate that such computer instructions can be written in a number of programming languages for use with many computer architectures or operating systems. Furthermore, such instructions may be stored in any memory device, such as semiconductor, magnetic, optical or other memory devices, and may be transmitted using any communications technology, such as optical, infrared, microwave, or other transmission technologies.

Among other ways, such a computer program product may be distributed as a removable medium with accompanying printed or electronic documentation (e.g., shrink-wrapped software), preloaded with a computer system (e.g., on system ROM or fixed disk), or distributed from a server or electronic bulletin board over the network (e.g., the Internet or World Wide Web). In fact, some embodiments may be implemented in a software-as-a-service model (“SAAS”) or cloud-computing model. Of course, some embodiments of the present disclosure may be implemented as a combination of both software (e.g., a computer program product) and hardware. Still other embodiments of the present disclosure are implemented as entirely hardware, or entirely software.

The devices and methods disclosed herein can be used in minimally-invasive surgery and/or open surgery. More specifically, the devices and methods can be used to probe or diagnose soft tissue such as skin, liver, vocal chords, colon or other soft tissue within the body. While the devices and methods disclosed herein are generally described in the context of a minimally-invasive procedure on a human patient, it will be appreciated that the methods and devices disclosed herein can be used in any of a variety of surgical procedures with any human or animal subject, or in non-surgical procedures, such as diagnostic procedures of tissue that has already been excised. Additionally, the devices and method disclosed herein can be used to measure and evaluate material properties of non-biological soft materials such as rubbers or gelatin.

The devices disclosed herein can be designed to be disposed of after a single use, or they can be designed to be used multiple times. In either case, however, the device can be reconditioned for reuse after at least one use. Reconditioning can include any combination of the steps of disassembly of the device, followed by cleaning or replacement of particular pieces, and subsequent reassembly. In particular, the device can be disassembled, and any number of the particular pieces or parts of the device can be selectively replaced or removed in any combination. Upon cleaning and/or replacement of particular parts, the device can be reassembled for subsequent use either at a reconditioning facility, or by a surgical team immediately prior to a surgical procedure. Those skilled in the art will appreciate that reconditioning of a device can utilize a variety of techniques for disassembly, cleaning/replacement, and reassembly. Use of such techniques, and the resulting reconditioned device, are all within the scope of the present application.

The devices described herein can be processed before use in a surgical procedure. First, a new or used instrument can be obtained and, if necessary, cleaned. The instrument can then be sterilized. In one sterilization technique, the instrument can be placed in a closed and sealed container, such as a plastic or TYVEK bag. The container and its contents can then be placed in a field of radiation that can penetrate the container, such as gamma radiation, x-rays, or high-energy electrons. The radiation can kill bacteria on the instrument and in the container. The sterilized instrument can then be stored in the sterile container. The sealed container can keep the instrument sterile until it is opened in the medical facility. Other forms of sterilization known in the art are also possible. This can include beta or other forms of radiation, ethylene oxide, steam, or a liquid bath (e.g., cold soak). Certain forms of sterilization may be better suited to use with different portions of the device due to the materials utilized, the presence of electrical components, etc.

It should be understood that the subject matter defined in the appended claims is not necessarily limited to the specific implementations described above. The specific implementations described above are disclosed as examples only.

Examples of the present disclosure include:
1. An injection device comprising:
   a tubular conduit having a deformable wall, the deformable wall defining a channel extending between a proximal end and a distal end of the tubular conduit, wherein the deformable wall comprises six sidewalls arranged as a convex hexagon and joined at angles by flexible joint regions between each of the six sidewalls, the six sidewalls comprising opposing and parallel first and second primary sidewalls and two opposing pairs of secondary sidewalls, the side sidewalls defining an perimeter of the channel;
   an inlet at a proximal end of the tubular conduit;
   an outlet at a distal end of the tubular conduit;
   a needle fluidly coupled with the outlet and configured to pierce into a soft material and deliver fluid from the tubular conduit to the soft material;
   an actuator comprising a first surface contacting the first primary sidewall of the tubular conduit; and
   a second surface contacting a second sidewall of the tubular conduit,
   wherein the actuator is configured to move a distance to deflect the tubular conduit by moving the first surface towards the second surface such that the angles at the flexible joint regions adjust to reduce the distance between the first primary sidewall and second primary sidewall, the tubular conduit configured to maintain a constant circumference of the perimeter during deflection of the first and second sidewalls, and
   wherein, upon deflection of the tubular conduit by the actuator, the outlet is configured to expel a micro-volume of an incompressible fluid from the channel against resisting pressure from the soft material.
2. The injection device of example 1, wherein the actuator is configured to move the first surface and the second surface simultaneously to reduce the distance between the first primary sidewall and the second primary sidewall.
3. The injection device of any of examples 1 to 2, wherein the first surface contacts the entire length of the first primary sidewall and the second surface contacts the entire length of the second primary sidewall.
4. The injection device of any of examples 1 to 3, wherein the conduit comprises a rigid wall region at the distal end before the outlet.
5. The injection device of any of examples 1 to 4, further comprising a controller configured to control the distance of the actuator based on a predetermined relationship between a distance of actuator movement and a micro-volume of fluid displaced from the tubular conduit.
6. The injection device of any of examples 1 to 5, further comprising a pressure sensor configured to measure pressure within the tubular conduit during an amount of time.
7. The injection device of example 6, wherein the pressure within the tubular conduit corresponds to a resisting pressure of the material at the outlet.
8. The injection device of any of examples 6 to 7, wherein the resisting pressure at the outlet is related to an expansion of a body of fluid at the outlet and at least one elastic property of the material.
9 The injection device of any of examples 1 to 8, further comprising a second conduit coupled to the inlet and configured to fill the tubular conduit while eliminating air from the fluid.
10. The injection device of any of examples 1 to 9, wherein the fluid exhibits laminar flow through the tubular conduit.
11. The injection device of any of examples 1 to 10, wherein the ratio of a length of the conduit and an inner radius of the conduit is greater than the fluid Reynolds number divided by 48.
12. A volume controlled injection system comprising:
   a tubular conduit having a deformable wall, the deformable wall defining a central channel extending between first and second ends of the tubular conduit, wherein the channel defines a volume suitable for containing a fluid therein, wherein the volume of the channel decreases upon exterior compression of the deformable wall;
   an actuator configured to compress the deformable wall by symmetrically impinging opposing sides of the deformable wall along an axis lateral to a central longitudinal axis of the tubular conduit; and
   a controller configured to control movement of the actuator,
   wherein the actuator is configured to move a distance to deflect the deformable wall toward the central longitudinal axis and reduce the volume of the channel such that a corresponding volume of fluid is displaced from the central channel through an outlet at the second end of the tubular conduit, and
   wherein the controller is configured to control the distance based on a predetermined relationship between actuator movement and a volume of fluid displaced from the tubular conduit.
13. The system of example 12, wherein the tubular conduit maintains a constant circumference of a perimeter of the channel during deflection of the opposing sides.
14. The system of any of example 12 to 13, wherein a change in volume of the channel relates to the volume of fluid displaced from the tubular conduit.
15. The system of any of examples 12 to 14, further comprising:
   a pressure transducer configured to measure pressure in the central channel, wherein the pressure in the central channel corresponds to a resisting pressure of the material at the outlet of the tubular conduit.
16. The system of any of examples 12 to 15, further comprising a housing having an inner cavity configured to house the tubular conduit, the housing comprising a handle for manual control and an opening covered by a spring-loaded cover.
17. The system of any of examples 12 to 16, wherein the actuator comprises a first plate and a second plate disposed on opposing sides of the tubular conduit, and a step motor configured to move the first plate towards the second plate to compress the tubular conduit between the first and second plates.
18. The system of any of examples 12 to 17, wherein the actuator comprises a second pressure sensor configured to measure pressure resulting from contact of the actuator against the deformable wall.
19. The system of example 18, wherein the second pressure sensor is a limit switch configured to limit the amount of actuator movement when a desired degree of sidewall deformation is reached.
20. The system of any of examples 12 to 19, wherein the tubular conduit comprises a region of high stiffness and a region of low stiffness and the actuator is configured to compress the region of low stiffness.
21. The system of any of examples 12 to 20, further comprising a power source configured to power the controller and the actuator.
22. The system of any of examples 12 to 21, wherein the volume of fluid displaced from the central channel is approximately in the range of about 1 nanoliter to about 1000 nanoliters.
23. The system of any of examples 12 to 22, wherein the volume of fluid displaced from the central channel is approximately in the range of about 1 microliter to about 1000 microliters.
24. A method for measuring elastic properties of soft material comprising:
   providing a conduit having a deformable wall defining a channel pre-filled with an incompressible fluid;
   inserting an outlet needle of the conduit into a soft material;
   deflecting an exterior portion of the deformable wall by displacement of an actuator to reduce the inner volume of the channel and introduce a body of incompressible fluid into the soft material from the conduit over an operation period of time;
   recording an internal pressure of the conduit over the operation period of time;
   determining an expansion of the body of incompressible fluid over the operation period of time based on a volume change of the body of incompressible fluid over the operation period of time; and
   determining an elastic property of the soft material based on a predetermined relationship between a displacement distance of the actuator, the expansion of the body of incompressible fluid, and the internal pressure of the conduit during the operation period of time.
25. The method of example 24, wherein the operation period of time extends from a start of actuator movement to at least a time when the body of incompressible fluid disperses into the soft material.
26. The method of any of examples 24 to 25, wherein the internal pressure of the conduit corresponds to the pressure of the soft material at the outlet.
27. The method of any of examples 24 to 26, wherein the deformable wall of the conduit is deflected symmetrically by the actuator.
28. The method of any of examples 24 to 27, wherein the deformable wall comprises a region of high stiffness and a region of low stiffness, and the actuator is configured to effect the compression upon the region of low stiffness.
29. The method of any of examples 24 to 28, wherein the actuator comprises a first plate and a second plate disposed on opposing sides of the conduit, and a step motor configured to move at least one of the first and second plates to compress the conduit between the first and second plates.
30. The method of any of examples 24 to 29, wherein the fluid is incompressible and devoid of air.
31. The method of any of examples 24 to 30, further comprises pre-filling the conduit by coupling a one way valve at an inlet of the conduit to a second conduit filled with the incompressible fluid, and the one-way valve is configured to eliminate bubbles from the conduit.
32. The method of any of examples 24 to 31, wherein the operation period of time is approximately in the range of about 0.01 seconds to about 1000 seconds.
33. The method of any of examples 24 to 32, wherein the material is a non-biological material.
34. The method of any of examples 24 to 33, wherein the soft material is a soft tissue.
35. The method of example 34, wherein the soft tissue is within a human patient.
36. The method of any of examples 34 to 35, wherein the soft tissue is thyroid tissue.
37. A method for measuring material properties of a soft material comprising:
   receiving by a processor, a first input comprising an increase in volume of a body of fluid introduced into the soft material over an operation period of time, wherein the body of fluid is spherical;
   receiving by a processor, a second input comprising a resisting pressure of the soft material over the operation period of time;
   determining a circumferential stretch of the body of fluid during the operation period of time based on the first input; and
   determining at least one material property of the soft material based on the circumferential stretch and the second input.
38. The method of example 37, wherein the operation period of time is from an initiation of the increase in volume of the body of fluid until a time when the body of fluid disperses throughout the soft material.
39. The method of example 37, wherein the at least one material property comprises at least one of elastic modulus, toughness, viscoelastic response or fracture progression.
40. The method of example 39, further comprising displaying a visual representation of the resisting pressure over the operation of time and the at least one material property on a visual display.
41. A system for measuring material properties of a soft material comprising:
   a tubular conduit having a deformable wall, the deformable wall defining a central channel extending between first and second ends of the tubular conduit, wherein the channel defines a volume suitable for containing a fluid therein;
   an actuator configured to move a distance along an axis lateral to a central longitudinal axis of the tubular conduit to deflect the deformable wall toward the central longitudinal axis and reduce the volume of the channel such that a corresponding volume of fluid is displaced from an outlet of the tubular conduit; and
   a controller configured to control movement of the actuator,
   wherein the controller is configured to control the distance based on a predetermined relationship between actuator movement and a volume of fluid displaced from the tubular conduit.
42. The system of example 41, wherein the tubular conduit comprises a rigid material with an opening exposing a deformable material.
43. The system of example 41, wherein the tubular conduit comprises a rigid material over molded on a flexible material.
44. The system of example 41, wherein the tubular conduit comprises a depressed region configured to receive the actuator.

One skilled in the art will appreciate further features and advantages of the disclosure based on the above-described embodiments. Accordingly, the disclosure is not to be limited by what has been particularly shown and described, except as indicated by the appended claims. All publications and references cited herein are expressly incorporated herein by reference in their entirety.

What is claimed is:

1. An injection device comprising:
a tubular conduit having a deformable wall, the deformable wall defining a channel extending between a proximal end and a distal end of the tubular conduit, wherein the deformable wall comprises six sidewalls arranged as a convex hexagon and joined at angles by flexible joint regions between each of the six sidewalls, the six sidewalls comprising opposing and parallel first and second primary sidewalls and two opposing pairs of secondary sidewalls, the side sidewalls defining an perimeter of the channel;
an inlet at a proximal end of the tubular conduit;
an outlet at a distal end of the tubular conduit;
a needle fluidly coupled with the outlet and configured to pierce into a soft material and deliver fluid from the tubular conduit to the soft material;
an actuator comprising a first surface contacting the first primary sidewall of the tubular conduit; and
a second surface contacting a second sidewall of the tubular conduit,
wherein the actuator is configured to move a distance to deflect the tubular conduit by moving the first surface towards the second surface such that the angles at the flexible joint regions adjust to reduce the distance between the first primary sidewall and second primary sidewall, the tubular conduit configured to maintain a constant circumference of the perimeter during deflection of the first and second sidewalls, and
wherein, upon deflection of the tubular conduit by the actuator, the outlet is configured to expel a micro-volume of an incompressible fluid from the channel against resisting pressure from the soft material.

2. The injection device of claim 1, wherein the actuator is configured to move the first surface and the second surface simultaneously to reduce the distance between the first primary sidewall and the second primary sidewall.

3. The injection device of claim 1, wherein the first surface contacts the entire length of the first primary sidewall and the second surface contacts the entire length of the second primary sidewall.

4. The injection device of claim 1, wherein the conduit comprises a rigid wall region at the distal end before the outlet.

5. The injection device of claim 1, further comprising a controller configured to control the distance of the actuator based on a predetermined relationship between a distance of actuator movement and a micro-volume of fluid displaced from the tubular conduit.

6. The injection device of claim 1, further comprising a pressure sensor configured to measure pressure within the tubular conduit during an amount of time.

7. The injection device of claim 6, wherein the pressure within the tubular conduit corresponds to a resisting pressure of the material at the outlet.

8. The injection device of claim 1, wherein the resisting pressure at the outlet is related to an expansion of a body of fluid at the outlet and at least one elastic property of the material.

9. A volume controlled injection system comprising:
a tubular conduit having a deformable wall, the deformable wall defining a central channel extending between first and second ends of the tubular conduit, wherein the channel defines a volume suitable for containing a fluid therein, wherein the volume of the channel decreases upon exterior compression of the deformable wall;
an actuator configured to compress the deformable wall by symmetrically impinging opposing sides of the deformable wall along an axis lateral to a central longitudinal axis of the tubular conduit; and
a controller configured to control movement of the actuator,
wherein the actuator is configured to move a distance to deflect the deformable wall toward the central longitudinal axis and reduce the volume of the channel such that a corresponding volume of fluid is displaced from the central channel through an outlet at the second end of the tubular conduit, and
wherein the controller is configured to control the distance based on a predetermined relationship between actuator movement and a volume of fluid displaced from the tubular conduit.

10. The system of claim 9, wherein the tubular conduit maintains a constant circumference of a perimeter of the channel during deflection of the opposing sides.

11. The system of claim 9, wherein a change in volume of the channel relates to the volume of fluid displaced from the tubular conduit.

12. The system of claim 9, further comprising:
a pressure transducer configured to measure pressure in the central channel,
wherein the pressure in the central channel corresponds to a resisting pressure of the material at the outlet of the tubular conduit.

13. The system of claim 9, further comprising a housing having an inner cavity configured to house the tubular conduit, the housing comprising a handle for manual control and an opening covered by a spring-loaded cover.

14. The system of claim 9, wherein the actuator comprises a first plate and a second plate disposed on opposing sides of the tubular conduit, and a step motor configured to move the first plate towards the second plate to compress the tubular conduit between the first and second plates.

15. The system of claim 9, wherein the tubular conduit comprises a region of high stiffness and a region of low stiffness and the actuator is configured to compress the region of low stiffness.

16. A method for measuring elastic properties of soft material comprising:
providing a conduit having a deformable wall defining a channel pre-filled with an incompressible fluid;
inserting an outlet needle of the conduit into a soft material;
deflecting an exterior portion of the deformable wall by displacement of an actuator to reduce the inner volume of the channel and introduce a body of incompressible fluid into the soft material from the conduit over an operation period of time;
recording an internal pressure of the conduit over the operation period of time;
determining an expansion of the body of incompressible fluid over the operation period of time based on a volume change of the body of incompressible fluid over the operation period of time; and
determining an elastic property of the soft material based on a predetermined relationship between a displacement distance of the actuator, the expansion of the body of incompressible fluid, and the internal pressure of the conduit during the operation period of time.

17. The method of claim 16, wherein the operation period of time extends from a start of actuator movement to at least a time when the body of incompressible fluid disperses into the soft material.

18. The method of claim 16, wherein the internal pressure of the conduit corresponds to the pressure of the soft material at the outlet.

19. The method of claim 16, wherein the actuator comprises a first plate and a second plate disposed on opposing sides of the conduit, and a step motor configured to move at least one of the first and second plates to compress the conduit between the first and second plates.

20. The method of claim 16, further comprises pre-filling the conduit by coupling a one way valve at an inlet of the conduit to a second conduit filled with the incompressible fluid, and the one-way valve is configured to eliminate bubbles from the conduit.
